(19) United States
(12) Patent Application Publication
(10) Pub. No.: US 2007/0179806 A1
(43) Pub. Date: Aug. 2, 2007

(54) Medication therapy management process

(76) Inventors: Calvin H. Knowlton, Philadelphia, PA (US); Douglas Joseph Weschules, Downingtown, PA (US); Brian Keith Esterly, Lansdowne, PA (US); Michael J. Groh, Mount Pleasant, SC (US); Thomas N. Wilson, Mount Pleasant, SC (US); Kevin Thomas Bain, Delanco, NJ (US)

Correspondence Address: MORGAN LEWIS & BOCKIUS LLP, 1111 PENNSYLVANIA AVENUE NW, WASHINGTON, DC, 20004, US

(21) Appl. No.: 11/344,211

(22) Filed: Feb. 1, 2006

(51) Int. Cl. G06Q 10/00; A61B 5/00

(52) U.S. Cl. 705/2; 600/300

(57) ABSTRACT

A medication therapy management process. The system receives input data relating to a patient profile including, but not limited to, a patients age, gender, and race; medical history; medication history; and problems such as allergies. The patient data is then compared to one or more databases according to a set of rules to produce a list of alerts that reduce the likelihood of medication misadventures.

### FIELD OF THE INVENTION

The present invention relates to a medication therapy management process. In particular, the present invention relates to a pharmacotherapy review that includes both medication and non-medication clinical data, and intervention services when potential medication-related problems (MRPs) are identified.

### BACKGROUND OF THE INVENTION

Older adults are particularly vulnerable to MRPs related to multiple, co-existing chronic illnesses that require complex drug regimens; sensory and motor deficits; cognitive impairment; and socio-economic challenges or barriers. If classified as a disease, MRPs would represent the fifth leading cause of death in the United States. MRPs include, but are not limited to, adverse drug events (ADEs), duplicate therapies and potentially inappropriate medications (PIMs). Despite the increased risk of hospitalization and death associated with PIM, the prevalence of PIM in the elderly ranges from 12-40%. Prevention or early identification of MRPs has the potential to significantly reduce MRP-associated morbidity, mortality, and economic costs. Tools for classifying vulnerable patients according to MRP risk are a necessary antecedent to development of effective interventions.

In medication distribution and selection systems, physicians prescribe, pharmacists dispense, and nurses administer and care for patients. Many healthcare providers have computerized information systems, which are typically stand-alone systems. Thus, a particular prescription decision may be at the mercy of one individual prescriber's clinical judgment, which may or may not reflect the most appropriate clinical judgment. This is further complicated by the fact that patients frequently have multiple physicians, and often, multiple pharmacies that, more likely than not, do not know what the others are prescribing or dispensing.

The appropriateness of drug therapy is dependent on many factors. Drug utilization review (DUR) is a process which pharmacists use to counsel consumers about such topics as the effects of taking two or more medications at the same time. When filling prescriptions, pharmacists generally check their customers' medication histories by using a computerized database created as a result of DUR efforts. However, these systems generally do not take into account other factors including, but not limited to, a patient's age, gender, and race; medical history; medication history; and problems such as allergies. Consideration of these factors is often as critical as avoiding adverse drug interactions.

Thus, it is believed that there is a need for efficient systems and methods of managing drug therapy by taking into account not only the possibilities of adverse drug interactions, but also factors including, but not limited to, patient demographics, medical history, medication history, and problems such as allergies. Such a system would provide benefits to patients such as an enhanced quality of life, increased control of debilitating symptoms, and reduction of adverse drug events; benefits to payors such as avoiding costly care and ensuring the right drug, right dose, and right frequency; and benefits to the prescriber such as ensuring adherence to best practices; the right drug, right dose, and right frequency, for the right patient; providing insight into clinically relevant data; providing verbal and written feedback; and increasing professional competence of clinician partners.

### SUMMARY OF PREFERRED EMBODIMENTS OF THE INVENTION

In one embodiment, the present invention is directed to a method for optimizing pharmacotherapy for a patient. Preferably, the method includes the steps of receiving data relating to a patient, identifying potential MRPs, assessing and indicating the status of each identified medication related problem, contacting a medication prescriber with recommendations, documenting the medication prescriber's response, and communicating recommendations and disposition of recommendations with partner(s). In this embodiment, potential MRPs are identified by comparing the data relating to the patient to an iterative database comprising medical reference data.

In another embodiment, the present invention is directed to a computer system for optimizing pharmacotherapy. Preferably, the computer system includes a database containing patient records, a database containing clinical rules to identify and detect potential MRPs and a processor that is used to identify potential MRPs that exist in a patient's current medication regimen and to prepare recommendations for a medication prescriber. In this embodiment, a processor that uses algorithms specific to a medication or medication class is used to review potential MRPs against patient records to prepare recommendations for a medication prescriber.

### DESCRIPTION OF THE DRAWINGS

FIG. 1 is a diagram of one embodiment of the present invention showing a medication therapy management system of the present invention;

FIG. 2 is a block diagram showing various components of the computer system of the present invention;

FIG. 3 is a block diagram showing the components of an integrated on-line interactive system of the present invention;

FIG. 4 is a diagram of one embodiment showing a therapy management process according to the present invention;

FIG. 5 is a diagram showing the process of FIG. 4 in greater detail;

FIG. 6 is a diagram depicting exemplary steps and components of a medications advisor module according to the process of FIG. 4;

FIG. 7 is a diagram depicting exemplary steps and components of a problems advisor module according to the process of FIG. 4;

FIG. 8 is a diagram depicting exemplary steps involved in a call from a prescriber or physician using the method of the present invention; and

FIG. 9 is a diagram depicting a first database process that is a step in the method depicted in FIG. 8.

### DETAILED DESCRIPTION OF THE PREFERRED EMBODIMENTS

Reference will now be made in detail to the preferred embodiments of the present invention, examples of which are illustrated in the accompanying drawings. Wherever possible, the same reference numbers will be used throughout the drawings to refer to the same or like parts and steps. The accompanying figures are illustrative, but not limiting, of the present invention.

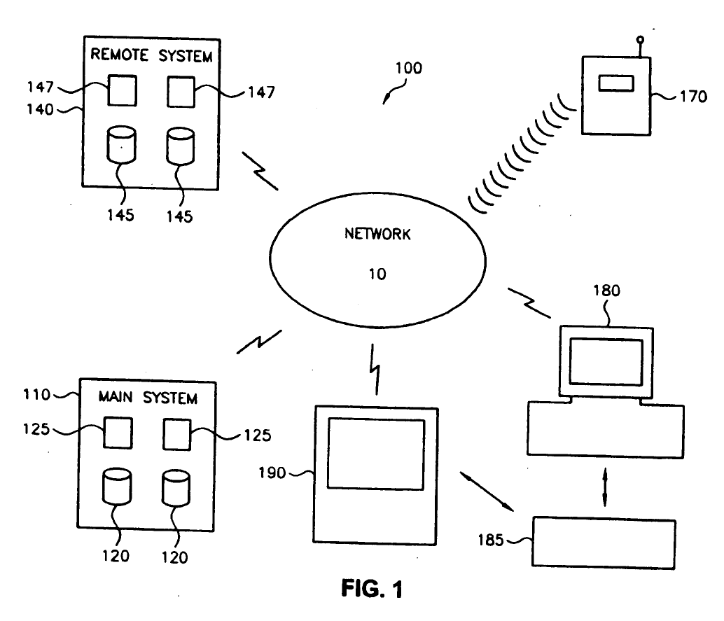

In accordance with one aspect of the present invention, a novel system and method for providing medication therapy management are provided. One embodiment of the present invention relating to a medication therapy management process, and pharmacotherapy review through a network system, is illustrated in FIG. 1. Network system 100 facilitates providing effective patient care by allowing caregivers to conduct traditional patient medication care activities, such as acquiring and using pertinent patient and medication information, prescribing and distributing medications, and monitoring patient medication uses. Monitoring patient medication can occur at any time and any place using any computer devices and the like, such as a personal computer or wireless Internet access device, including hand-held devices.

In particular, network system 100 can be used, among other things, to integrate decentralized medication therapy management processes into a shared, centralized, controlled environment. More specifically, network system 100 serves to integrate the collection processes of patient data, medication trial data, actual patient treatment outcome data, and other relevant clinical data by bringing caregivers into a shared, centralized, controlled environment. Using the integrated collection of patient data and medication data, network system 100 can be used to improve medication prescribing and dispensing decisions. The improved decisions, in turn, promote, among others, the safety and efficacy of patient medication uses.

As discussed in detail below, network system 100 includes specialized databases that include patient profiles and other evidence-based pharmacotherapy data that enable pharmacists to provide recommendations to prescribers. In particular, network system 100 allows the pharmacist to identify potential MRPs in a particular patient who is using a particular medication for a particular medical indication. In other words, using network system 100, pharmacists can make accurate recommendations by using evidence-based guidelines to ensure the patient's medication regimen is most appropriate.

As shown in FIG. 1, it should be apparent that, using network 10, caregivers and pharmacists can remotely access network system 100 securely, in real time, by using any electronic communication media such as personal computer 180 or Personal Digital Assistant 170. Those skilled in the art will understand that any user interface may be used to input data, including but not limited to a keyboard, mouse, and other peripheral inputting devices of a computer system. Note that personal computer 180 or any other electronic communication media can be connected to displaying devices such as a monitor or printing device, such as printer 185. Using printer 185, all exchanged information over Network 10 can be printed in hard copies. Personal computer 180 can be located anywhere, including the caregiver's home or office. Also, portable, wireless Internet access devices such as Personal Digital Assistant 170 can be used to access network system 100 remotely via Network 10. Once connected to Network 10, Personal Digital Assistant 170 or personal computer 180 can be used to connect to Main System 110 and Remote System 140. Note that Network 10 can be any type of network systems such as a Local Area Network, Wide Area Network, or a global network such as the Internet.

Those skilled in the art will understand that network system 100 may receive communication signals over any suitable medium such as twisted-pair wire, co-axial cable, fiber optics, radio-frequencies, and so forth.

Network system 100 includes Main System 110, which, as described more in detail below, includes one or more processors 125. Main System 110 can be any commercially available computer system such as a server, minicomputer or microcomputer, mainframe, and the like. Main System 110 further includes one or more specialized databases 120 for storing, among other things, patient data, medical reference data, clinical rules, and Medication Use Guidelines (MUGs™) data. MUGs™ are proprietary step-care algorithms, which are derived from evidence-based literature, clinical experience, standards of practice, and an extensive database of medical information. As shown, one or more processors 125 may be used in connection with executing a number of different computer programs or software applications in carrying out the methods of the present invention. Main System 110, in accordance with one aspect of the present invention, is preferably located at a main facility such as a central data management facility.

In one embodiment of the present invention, Main System 110 is located at a centralized contact center (or call center) equipped with on-site pharmacists. Pharmacists can perform medication therapy management to support the prescriber and dispense, if necessary, from one facility.

Network system 100 further includes one or more Remote Systems 140, which are operatively connected to Main System 110 via a global network such as the Internet. As shown in FIG. 1, Remote System 140 is connected to network system 100 via network 10. Remote System 140 is similar to Main System 110. Remote System 140 can be a computer system having one or more processors 147 and one or more specialized databases 145. In accordance with one embodiment of the present invention, Remote System 140 is preferably located at one or more medication care facilities such as a hospital, pharmacy, hospice, and medication dispensing center.

It should be apparent from the foregoing description that processors 125 can access databases 120 using local links such as a bus system. Using network 10, processors 125 may also access databases 145 of Remote System 140. Like databases 120 of Main System 110, databases 145 may store at least one of patient data, medical reference data, and MUGs™ data. Thus, processors 125 may access all files included in databases 145 via network 10 and look up data, in addition to data stored in databases 120, as needed in carrying out methods of the present invention. Likewise, processors 147 can access databases 145 within Remote System 140 using its local links, or alternatively and/or additionally, processors 147 can access databases 120 of Main System 110 via network 10. Of course, processors 147 of a Remote System 140 can also access databases 145 of another Remote System 140 via network 10. In one embodiment, the data files are shared through a file transfer on a daily basis.

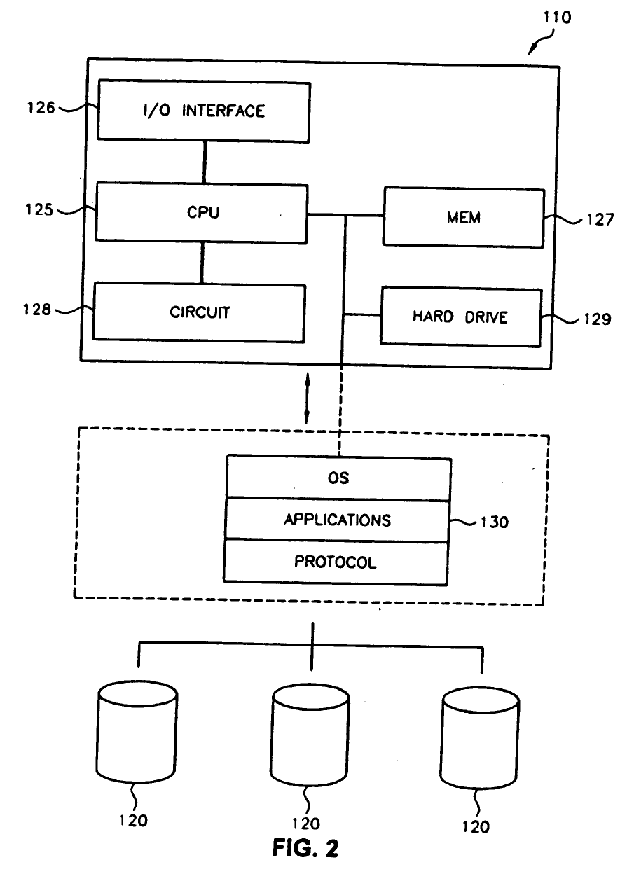

FIG. 2 shows one embodiment of components of Main System 110, which can be any commercially available computer system, such as a server, mainframe, or microcomputer. As illustrated earlier, Main System 110 includes at least one processor (or CPU) 125. Processor 125 is operable with, among other things, a main memory 127, an input/output (I/O) device 126, and such well-known support circuits 128 as power supplies, clocks, caches, displays, and the like. I/O device 126 receives and transmits electrical signals corresponding to an electrical signal that passes over network 10. Main memory 127 includes instructions that processor 125 executes to facilitate the processing, storage, transfer, and control of data transfer and storage. Instructions in memory 127 are in the form of program code. Main System 110 further comprises hard drive 129 for storing computer programs or application software, in accordance with one aspect of the present invention. Operating system software, application software, and other intelligent protocols or modules, collectively referred to as program modules 130, are stored in hard drive 129 and main memory 127 of Main System 110. Using instructions of modules 130, processors 125 communicate with databases 120. As discussed in detail below, using modules 130 and databases 120, in accordance with the present invention, novel ways of collecting and storing patient data and medication data, accessing and using patient data and medication data, and predicting potential MRPs of administering selected medications to patients are provided.

Like Main System 110, one or more Remote Systems 140, and other computer systems such as personal computer 180 that interface with network 10, may also be such servers or microcomputers capable of communicating over a computer network. Accordingly, program modules 130 may also be located in Remote Systems 140, or personal computer 180, and provide the same or similar functionality and utility as Main System 110. Those of ordinary skill in the art will recognize network system 100 (shown in FIG. 1) may connect to any number of additional computer systems that are capable of providing the functions of Main System 110.

Referring again to FIG. 1, Main System 110 includes one or more databases 120 that facilitate carrying out the methods of the present invention. Databases 120 include, as described further below, a patient database. The patient database comprises information relating to patients profiled in network system 100. Preferably, the patient database comprises information from all patients ever profiled in network system 100 and each patient profile is comprehensive, i.e., it includes all relevant patient and/or medication records. Thus, the term “profiling” is used to describe the process of recording what a patient is taking (i.e., what was already prescribed and dispensed as well as the over-the-counter products). Using network system 100, in accordance with one embodiment of the present invention, the source of the information that forms the basis of patient profile comes from one or more sources including the patient, pharmacy, hospice, hospital, lab, nurses, physicians, etc. The patient database of the present invention is preferably comprehensive.

As described in detail below, the comprehensive patient database, as used in this disclosure, includes information representing both objective attributes and subjective attributes. In accordance with the present invention, the term “objective” is used to refer to those attributes that are readily observable or measurable, and that can be easily compared among all patients. The objective attributes of the patient database include, for instance, the patient's gender, which can be easily compared from one patient to another. The term “subjective” is used to refer to those attributes that may not be equally applicable to all patients. The subjective attributes define, for instance, a patient's pain level, mobility, or personal satisfaction with a particular treatment or medication. The subjective attributes may also include an individualized result of treatment—e.g., the measure of how well a particular medication worked when administered to a patient having a particular symptom. These subjective attributes may not be easily compared from one patient to another. In other words, the subjective attributes define a “quality of life” of a patient by quantifying otherwise immeasurable factors.

Accordingly, the patient database includes, among others, objective patient profile attributes such as patient's demographic profile and medical history, all tailored to each patient. Medical history includes all pertinent medical information such as the patient's treating physician information, medication history including current prescription and over-the-counter medications, lab results, generic history, hospital and hospice records, recent diagnosis, existing allergy, etc. Medical history may also include a physician's (or any other qualified caretaker's) observation of using a particular medication on a patient. Demographic profile includes all other relevant information such as patient's age, contact information, race, geographic information, etc. The patient database also includes the subjective patient profile attributes such as the pain level indicated by the patient and the pain level diagnosed by a treating physician. The subjective attributes further include a patient's opinion, such as one's satisfaction, regarding using a particular medication. It should be apparent from the foregoing description that the patient database of the present invention represents a unique combination of both patient inputs and non-patient inputs.

Databases 120 also include a general medical reference database. The medical reference database is a database containing relevant medication and therapeutic information. In one embodiment of the present invention, the medical reference database includes First Data Bank (FDB) database, which includes descriptive, economic and clinical information relating to over 200,000 drug products. As noted earlier, databases 120 may further include a MUGs™ database. The MUGs™ database, in accordance with one aspect of the present invention, includes evidence-based and clinician-based, clinical trial results of selected medications that serve as a guide for prescribing medication for certain medical indications.

The MUGs™ database further includes peer-reviewed, step-care protocols relating to all relevant aspects of selected medications. The relevant aspects include, among other things, the efficacy, safety including any side effects, long term effect, cost information, and patient's unique and general response or reaction to selected medications. For instance, some of the protocols included in the MUGs™ database may show the efficacy and safety of medications and treatments relating to Congestive Heart Failure, End Stage Renal Disease, and Alzheimer's Disease.

Furthermore, the MUGs™ database can be organized into multiple representations. For instance, the MUGs™ database can organize selected medications based on their efficacy relating to particular indications. In one embodiment, selected medications within the protocols of the MUGs™ database are sorted by diagnosis coverage code in the index. In yet another embodiment, a brand and generic list of medications of over 75 compounds and an injectable medications list are included in the MUGs™ database. It should be noted that the MUGs™ database is dynamic; it is constantly updated by a medical professional committee to reflect new findings and guidelines relating to selected medications, diseases/conditions, and symptoms.

In accordance with one aspect of the present invention, databases 120, 145 include a database system using a query language such as Structured Query Language (SQL) database. An SQL database system can be used to extract data from databases 120, 145. An SQL database system facilitates the utility and functionality of network system 100 since, in one embodiment of the present invention, databases 120 and 145 are spread out over two or more computer systems over network system 100. Using a SQL database system allows multiple caregivers on network system 100 to simultaneously access databases 120, 145.

In accordance with another aspect of the present invention, databases 120, 145 are iterative. That is, databases 120, 145 may comprise an iterative database of empirical data on the effects of medication therapies on a plurality of patients whereby the patients can be stratified based on patient profile parameters, including subjective and/or objective attribute. Databases 120, 145 are updated after each access.

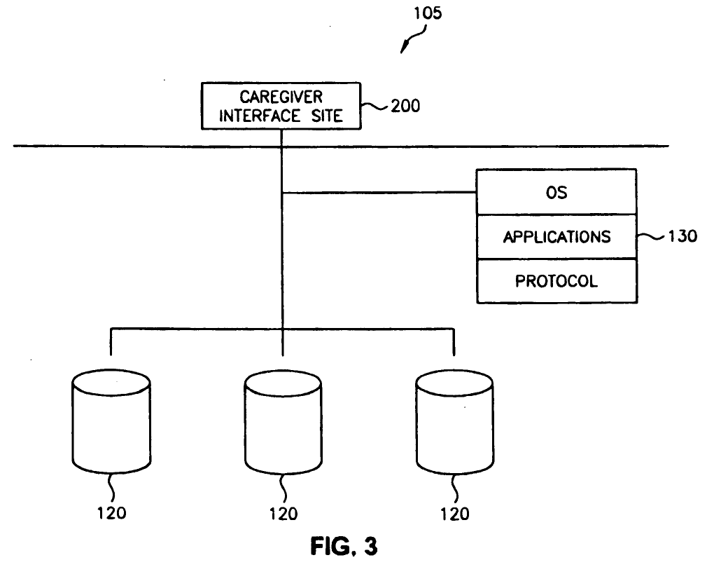

FIG. 3 shows one aspect of the present invention illustrating a novel system and method of using network system 100 called Predictive Pharmacotherapy Outcome System (PPOS) 105. PPOS 105, shown in FIG. 3, comprises major components of Main System 110. Thus, PPOS 105 includes program modules 130 and databases 120. In one embodiment as shown in FIG. 3, PPOS 105 represents a logical construction of an integrated, online caregiver interactive interface 200 using Main System 110 according to the present invention. As shown, PPOS 105 includes caregiver interface site 200. Caregiver interface site 200 represents one aspect of active server pages (ASP) that are accessed using an interactive programming language or forum such as an Intranet or Extranet site, or a query program such as Microsoft's Analysis Services. In one embodiment, caregiver interface site 200 comprises a dynamically created web page site that utilizes Object Linking and Embedding (OLE) or Component Object Model (COM) technologies such as ActiveX scripting—usually VB Script or Jscript code.

Caregiver interface site 200 is used in many aspects to carry out the methods of the present invention. For instance, caregiver interface site 200 can be used to facilitate information exchange between and among caregivers, Main System 110, and Remote System 140. Caregivers, using a network browser on their personal computers or wireless, hand-held Internet devices, request caregiver interface site 200, and then Main System 110 generates a page with web-based authoring tools such as HTML (or XML) code and sends it back to the browser. In effect, in accordance with one aspect of the present invention, caregiver interface site 200 represents a centralized server site for caregivers to, among other things, conduct all relevant communications between and among each other, Main System 110, and Remote System 140. Thus, PPOS 105, while using caregiver interface site 200, is used to carry out multiple embodiments of the present invention.

As noted earlier, in one embodiment network system 100 facilitates providing effective patient care by allowing caregivers to conduct traditional patient medication care activities, such as acquiring and using all pertinent patient information, prescribing and distributing medications, and monitoring patient medication uses, etc., electronically at any time and any place using any computer devices and the like such as a personal computer or wireless Internet access device including hand-held devices. Caregiver interface site 200 provides one aspect of the present invention that facilitates interactions between caregivers and network system 100. Thus, caregivers use caregiver interface site 200 to submit input data to network system 100 and to receive output data from network system 100.

For instance, in the embodiment shown in FIG. 1, caregivers log in to Main System 110 using personal computer 180 via network 10. Once logged in to Main System 110, a caregiver will be directed to caregiver interface site 200 as shown in FIG. 3. Caregiver interface site 200 can be used by authorized caregivers to access sensitive and/or privileged information stored in databases 120, 145. Therefore, in one embodiment of the present invention, after a caregiver has logged into Main System 110 via network 10, it is necessary to enter a caregiver identification number along with a password. Thereafter, with the aid of program modules 130 of Main System 110 or Remote System 140, the caregiver can access the contents of databases 120, 145.

It should be apparent that the present invention provides a secure environment for these caregiver interactions. In all embodiments of the present invention, the system includes software and hardware that can be used to secure all data and transactions in the present invention. For example, all data and information transmitted and received using network system 100, and stored in Main System 110 or Remote System 140 may be encrypted and/or password (or access code) protected. Further, any user's (e.g., caregiver's) access may be restricted to certain data and certain information by appropriate password (or access code) and/or encryption protection. The present invention meets all the requirements of the Health Insurance Portability and Accountability Act of 1996 (HIPAA).

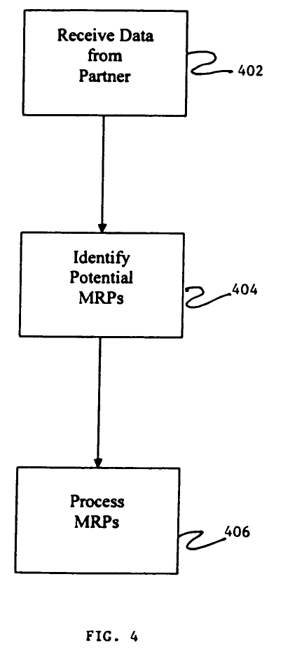

In accordance with one aspect of the present invention, a novel system and method for medication therapy management is provided. One embodiment of the present invention relating to medication therapy management is illustrated in FIG. 4.

In particular, the process of FIG. 4 is, among other things, a systematic way of identifying potential MRPs and assigning a risk to them to assist in prioritization. A pharmacist may intervene to develop a recommendation and communicate this recommendation to a physician prescriber, and then document the intervention in the same system that identified the problem.

FIG. 4 shows the three main steps involved in one embodiment of the present invention. Step 402 involves receiving data concerning a patient from a partner such as a payer or case manager. This step may include, but is not limited to, enrolling members to create their profiles, follow-up at predefined frequency, and processing inbound calls. This information is used to create a patient profile and comprises information such as demographics (e.g., age, gender, and race), medical history, medication history, and risk factors (e.g., allergies) peculiar to the patient.

In the second step 404, potential MRPs are identified and the patient is stratified into a risk group.

In the third step 406, the MRPs identified in step 404 are processed. Identified MRPs are prioritized. A pharmacist reviews the patient's chart and identified MRPs, assesses and indicates the status of each MRP, and prepares a recommendation for the prescriber. The pharmacist contacts the prescriber and reviews the recommendations. The prescriber's response is then documented and prepared for transmission back to the partner pharmacist.

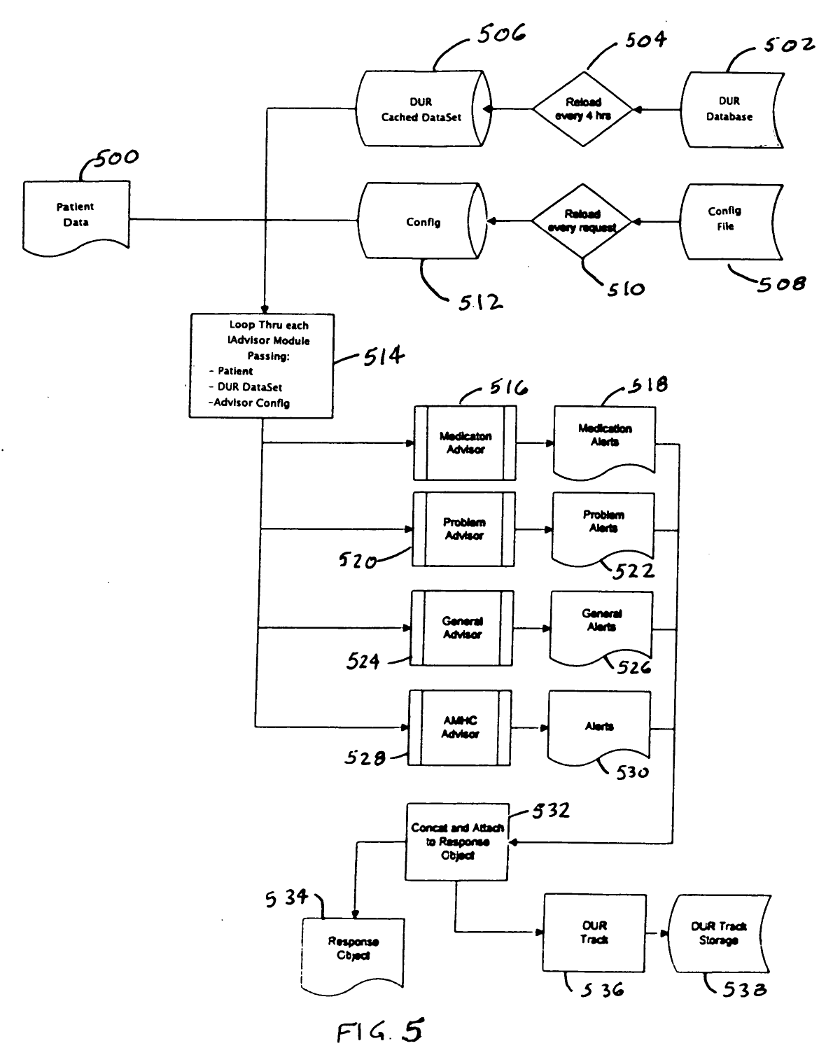

FIG. 5 shows a more detailed diagram of the process shown in FIG. 4. Block 500 represents patient data being input to the system. Block 502 represents a DUR database, which is reloaded in step 504 into the DUR cached data set 506. Block 508 represents Config File, XML based configuration files that allow the system to be configured in several different ways. For example, the rules data could be stored in several types of data stores (SQL Server, Oracle, MySql, XML, etc.). Config File 508 tells the system which data store to use and how to access it. Config File 508 also specifies the modules the system should execute against the patient XML record, or the system could be configured to exclude certain modules as well. Config File 508, as shown in step 510, is reloaded onto Config 512 each time a request is entered into the system. In step 514, the patient data, the DUR data set, and Config File 508 are looped through each advisor module to identify potential MRPs and to stratify a patient into a risk group. The instructions of the process may include algorithms which are used to compare an individual patient profile and medical indication of that patient to the profiles of all other patients in the DUR database 502. The advisor modules include the medication advisor module 516, the problem advisor module 520, the general advisor module 524, and the partner advisor 528. Those advisor modules produce medication alerts 518, problem alerts 522, general alerts 526, and customer alerts 530 by processing the patient data and the DUR dataset. The alerts are then concatenated in step 532 and attached to response object 534, and sent along DUR track 536 to DUR track storage. DUR track storage is a data warehouse which logs all generated alerts for tuning and modeling. DUR track storage logs certain characteristics of the patient and attaches the generated alert. Modeling and tuning are performed using analytical software to generate predictive models and/or outcomes.

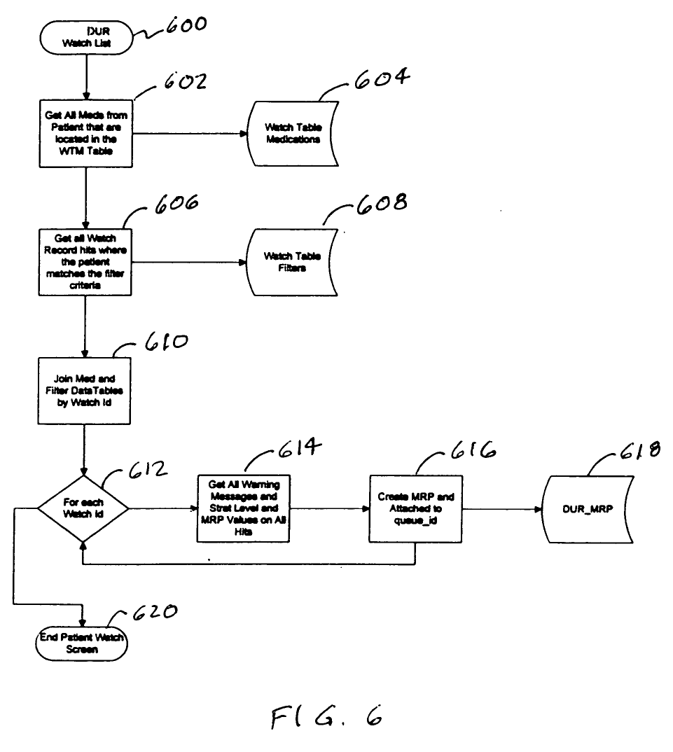

FIG. 6 shows the steps and components of the medications advisor module 516 of FIG. 5. This module uses a patient's medication list as watch medications to compare against a defined filter, and if a match occurs a medication alert is generated. MRPs are determined using rules developed specific to a medication or medication class present on a patient's medication profile. Targeted medication categories include, but are not limited to, narrow therapeutic index medications, potentially inappropriate medications, medications with a defined therapeutic range associated with optimal outcomes, medications with a large number of potential drug-drug interactions, medications requiring therapeutic drug monitoring, and medications with drug-disease interactions. The DUR watch list 600 is a list of medications defined within the medications advisor module. DUR watch list 600 provides a database against which the patient's medications 604 are compared in step 602. In step 606, the patient's medication information is compared to watch table filters 608. Those filters can be any number of filters comprising, but not limited to ICD-9 (International Classification of Disease) codes, dose, medication, gender, race, and dosage form. The filters can contain the compare types equals (=), greater than (>), less than (<), greater than or equals (>=), and not equals (!=). The filters can also have and/or operators that instruct the module on how one filter relates to other filters. The information from both the DUR watch list and the watch table filters in step 610 and watch IDs are created. At 612, a loop is begun that for each watch ID, goes through steps 614 (get all messages, and stratification level and MRP values) and 616 (create MRP and attach to queue_id). The results of steps 612, 614, and 616 are produced as a list of DUR MRPs and the patient watch screen is ended in step 620 when the loop is completed for each watch ID.

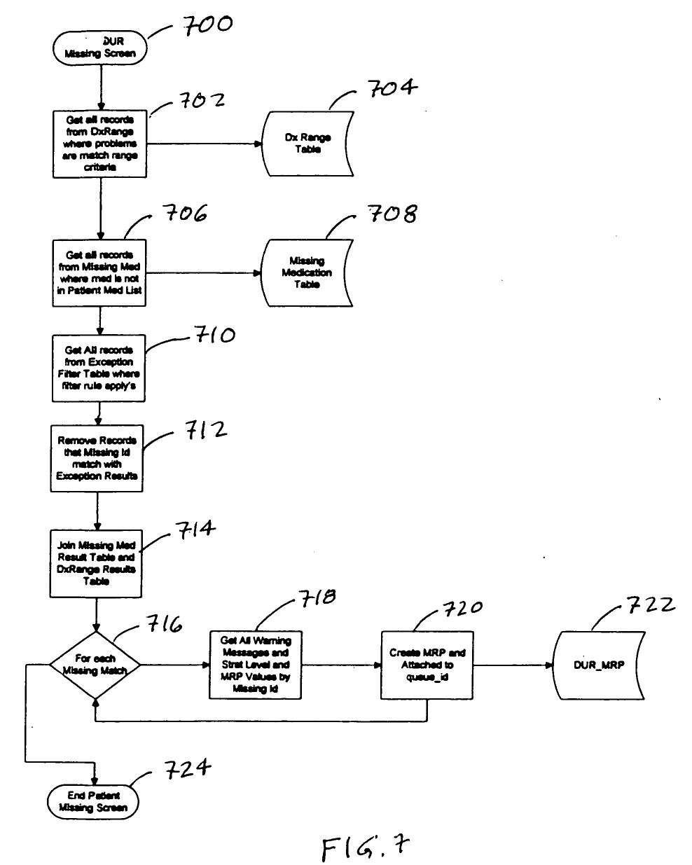

FIG. 7 shows the steps and components of the problems advisor module 520 of FIG. 5. This module looks for missed medications for reported problems. The rules are focused on missing drug therapy associated with favorable outcomes. The reported problems fall within a diagnosis range (“Dx range”) as indicated by an ICD-9 code or range of codes associated with that diagnosis or symptom. For example, depression has an ICD-9 of 311, whereas chronic heart failure has a diagnosis range of 428.0-428.9. If a patient has a problem for which no medication has been prescribed, the module produces an alert. Upon opening a DUR missing screen 700, all records are obtained from the Dx range table 704 in step 702. In step 706, all records are obtained from the missing medication table 708 where the medication is not in the patient's medication list. Steps 712 and 714 make up the exception filter process, in which if a patient meets filter criteria for missing medications, an alert is generated. The alert may be in the form of “No [appropriate medication] for [patient problem].” As with the medications advisor module 516, in the problems advisor module, the filters can contain the compare types equals (=), greater than (>), less than (<), greater than or equals (>=), and not equals (!=). The filters can also have and/or operators that instruct the module on how one filter relates to other filters. Step 716 begins a loop in which all warning messages, and stratification level and MRP values are obtained by missing IDs in step 718 and the MRP created and attached to queue_id in step 720. The results of steps 716, 720, and 722 are produced as a list of DUR MRPs and the patient missing screen is ended in step 724 when the loop is completed for each missing match.

The general advisor module 524 of FIG. 5 may allow a system manager to specify general rules using filters. General rules are a set of rules that are applied to all patients to help identify those at high risk for medication misadventuring. General rules include, but are not limited to number of medications per day, number of doses per day, number of co-morbidities, number of physicians, and number of pharmacies. More general rules can be added as requested, The system manager may be able to modify the compare type and the comparison variables, which will generate an alert.

The customer advisor module 528 of FIG. 5 may be customized for each customer. Custom tables may include, but are not limited to assessments, past procedures, and contraindications.

Once a list of MRPs is produced, a pharmacist reviews information available, collects more information as necessary, and develops pharmacotherapy recommendations. These recommendations may include, but not be limited to, optimizing therapy, discontinuing medications, changing medications, and initiating new medications. A comprehensive medication profile along with therapeutic recommendations may then be communicated to a physician responsible for patient care. A physician may then assess these recommendations along with the patient's reported medication profile, and act on the pharmacotherapy recommendations and initiate any suggested changes. The recommendations and changes may then be communicated to the nurse manager. The patient and caregiver may then confer to confirm current medication regimen accuracy, address questions, concerns, and issues associated with the medication regimen, and reinforce patient reported outcome measures tracking.

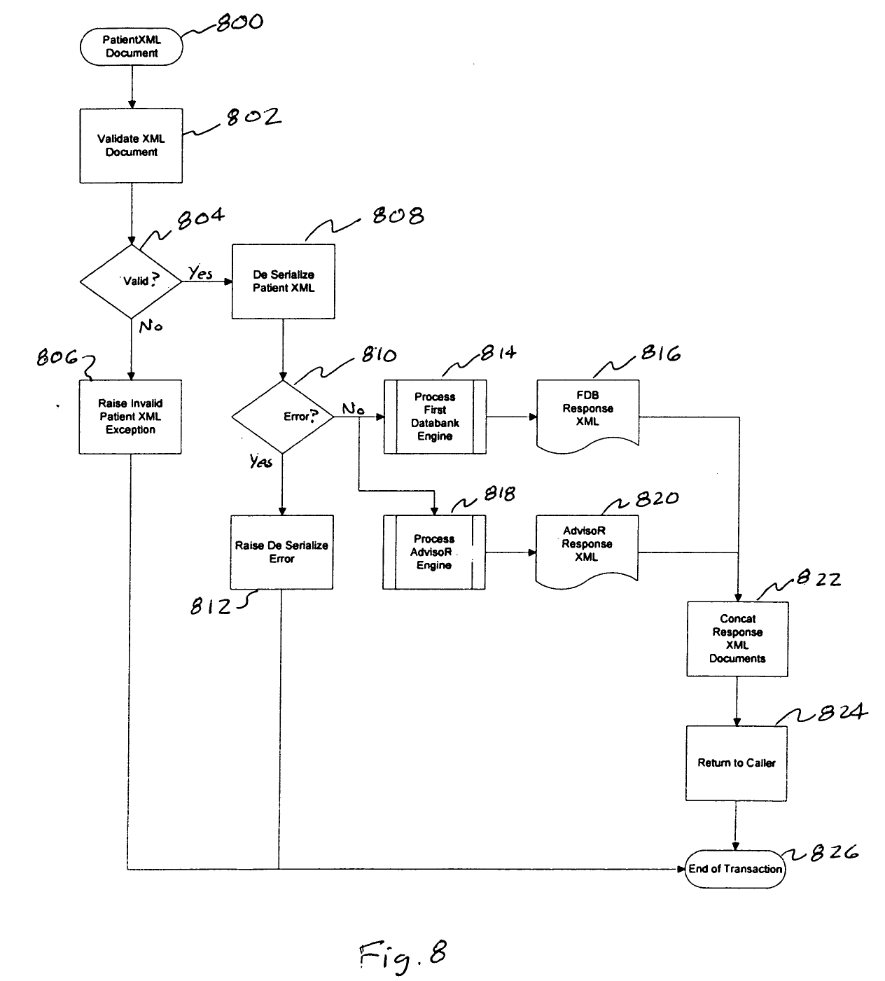

The process of taking a call transaction concerning a patient in one embodiment of the invention may be shown by FIG. 8. Upon receiving a call from a caregiver, nurse, or physician, an operator would bring up a XML document for the patient in step 800. The operator would then attempt to validate the XML document in step 802. An XML document is considered invalid if the document is not in the required format or all of the required data elements do not exist. In step 804, the validation status of the XML document would determine the next step of the process. If the XML document is determined to be invalid (e.g., the record contains an incorrect social security number or name misspelling), an invalid patient XML exception is raised in step 806 and the transaction ends at step 826. If the XML document is determined to be valid, the patient XML is deserialized in step 808. In step 810, the XML document is checked for deserialization error. If there is an error deserializing the XML document in step 808, a deserialization error is raised in step 812 and the transaction ends at step 826. If no deserialization error is detected, the process first database (FDB) engine and the process DUR engine run concurrently in steps 814 and 818, respectively and produce FDB response and DUR response XML documents in steps 816 and 820, respectively. The response XML documents are then concatenated in step 822 before returning to the caller in step 824 to end the transaction in step 826.

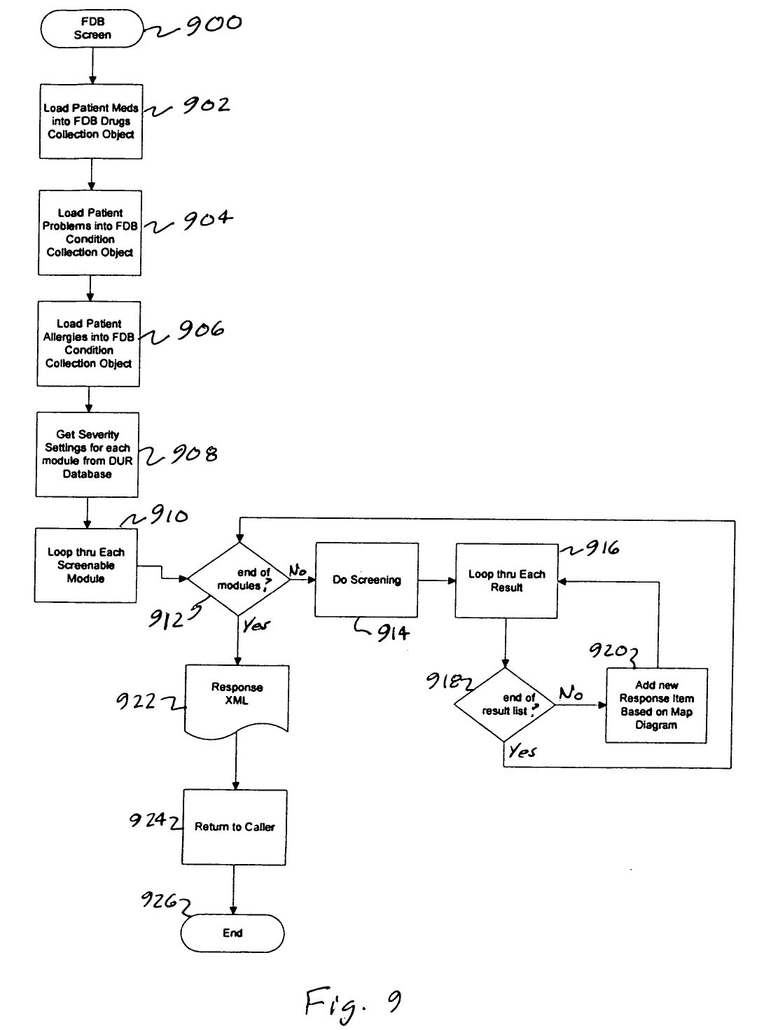

The FDB process is diagrammed in FIG. 9. In step 900, a FDB screen is presented. From this screen, patient medications are loaded into an FDB drugs collection object in step 902, patient problems may be loaded into an FDB condition collection object in step 904, and patient allergies may be loaded into the FDB condition object in step 906. The system may then provide severity settings for each module from the DUR database in step 910. Each screenable module is looped through in step 910. In step 912, a determination is made whether the end of modules has been reached. If the end of modules has been reached, a response XML document is produced in step 922 (corresponds to steps 816 and 822 in FIG. 8), the operator returns to the caller in step 924 (corresponds to step 824 in FIG. 8), and the transaction ends in step 926 (corresponds to step 826 in FIG. 8). If the end of modules has not been reached in step 912, screening is performed in step 914, with each result looped through in step 916. In step 918, a determination is made as to whether the end of the result list has been reached. If so, the process returns to step 912, at which point a determination is made as to whether the end of modules has been reached. If in step 918, a determination is made that the end of result list has not been reached, a new response item based on the map diagram of FIG. 9 is added and the process returned to step 916.

While the description herein refers to the information in multiple databases, those of ordinary skill in the art will recognize and understand that all such information could be stored in a single database or in several databases structured differently than those described herein,

Furthermore, the systems and methods of the present invention are equally applicable to patients in any health care system including, but not limited to, a hospital, clinic, long-term care facility, nursing home, patient's home, and may be used for inpatient and outpatient care.

It will be appreciated by those skilled in the art that changes could be made to the embodiments described above without departing from the broad inventive concept thereof. It is understood, therefore, that this invention is not limited to the particular embodiments disclosed, but is intended to cover modifications within the spirit and scope of the present invention as defined in the appended claims.

What is claimed is:

1. A method for optimizing pharmacotherapy for a patient, comprising:
receiving data relating to a patient;
identifying potential medication-related problems;
assessing and indicating the status of each identified medication related problem;
contacting a medication prescriber with recommendations; and
documenting the medication prescriber's response;
wherein potential medication-related problems are identified by comparing the data relating to the patient to an iterative database comprising medical reference data.

2. The method of claim 1, wherein the data relating to a patient includes at least one of the following:
demographic information;
medical history;
medication history; and
risk factors.

3. The method of claim 1, further comprising prioritization of a queue of potential medication-related problems.

4. The method of claim 1, further comprising stratification of the patient into a risk group.

5. The method of claim 1, wherein recommendations to medication prescribers comprise one of the following:
medication alerts;
problem alerts;
general alerts;
customer alerts; and
messages.

6. The method of claim 1, wherein rules are developed specific to a medication or medication class present in the data relating to a patient.

7. The method of claim 1, wherein medications are divided into targeted medication categories comprising:
narrow therapeutic index medications;
potentially inappropriate medications;
medications with a defined therapeutic range associated with optimal outcomes;
medications with a large number of potential drug-drug interactions;
medications requiring therapeutic drug monitoring; and
medications with drug-disease interactions.

8. The method of claim 1, wherein the recommendations to a medication prescriber include problem alerts.

9. A computer system for optimizing pharmacotherapy, comprising:
a database containing patient records;
an iterative database containing potential medication-related problems; and
a processor that uses algorithms specific to a medication or medication class to review potential medication-related problems against patient records to prepare recommendations for a medication prescriber.

10. The computer system of claim 9, wherein the patient records include at least one of the following:
demographic information;
medical history;
medication history; and
risk factors.

11. The computer system of claim 9, wherein an output is a prioritized queue of potential medication-related problems.

12. The computer system of claim 9, wherein an output is a list of patients stratified into risk groups.

13. The computer system of claim 9, wherein the recommendations comprise one of the following:
medication alerts;
problem alerts;
general alerts;
customer alerts; and
messages.

14. The computer system of claim 9, wherein rules are developed specific to a medication or medication class present in the data relating to a patient.

15. The computer system of claim 9, wherein medications are divided into targeted medication categories comprising:
narrow therapeutic index medications;
potentially inappropriate medications;
medications with a defined therapeutic range associated with optimal outcomes;
medications with a large number of potential drug-drug interactions;
medications requiring therapeutic drug monitoring; and
medications with drug-disease interactions.

16. The computer system of claim 9, wherein the recommendations to a medication prescriber include problem alerts.
